(19) United States
(12) Patent Application Publication
(10) Pub. No.: US 2024/0091554 A1
(43) Pub. Date: Mar. 21, 2024

(54) BETA CONVERTER SYSTEM AND METHODS OF USE

(71) Applicant: Havas Health, Inc., New York, NY (US)

(72) Inventors: Allison Ceraso, Rockville Centre, NY (US); Takae Takahashi, Long Island City, NY (US); Matt Franowicz, West Orange, NJ (US)

(21) Appl. No.: 18/514,439

(22) Filed: Nov. 20, 2023

Related U.S. Application Data
(63) Continuation of application No. PCT/US2022/030285, filed on May 20, 2022.
(60) Provisional application No. 63/191,550, filed on May 21, 2021.

(51) Int. Cl. A61N 5/06

(52) U.S. Cl. CPC A61N 5/0618; A61N 5/0622; A61N 2005/0626; A61N 2005/0662

(57) ABSTRACT

Provided herein are systems and methods and for a beta converter to treat Alzheimer's disease and increase patience adherence.

### CROSS REFERENCE TO RELATED APPLICATIONS

This application claims priority to PCT application serial no PCT/US2022/030285 filed May 20, 2022, which claims priority to U.S. provisional application Ser. No. 63/191,550, filed May 21, 2021, each herein incorporated by reference in their entireties.

### BACKGROUND

This invention generally to the therapeutic treatment of Beta Amyloid plaques disrupting neuronal activity in the brain, possibly causing the gradual loss of memory and cognitive function in people with Alzheimer's disease.

Gamma activity (e.g., 20-80 Hz neural activity) has long been theorized to facilitate neural communication, synaptic plasticity, coding of information across many neurons (neural codes) by driving cells to fire together on short timescales. Recently, a surprising new role for gamma activity was discovered: driving gamma frequency activity recruits microglial engulfment and clearance of amyloid beta (Ab) in an Alzheimer's Disease (AD) mouse models. Importantly, gamma attenuation was found prior to behavioral deficits, cell death and significant plaque buildup in an Alzheimer's mouse model, suggesting that gamma deficits are an integral component of AD pathology.

Preliminary research suggests LED lights flickering at a specific frequency can substantially reduce beta amyloid plaques. Currently, it is difficult to get patients to adhere to light therapy protocols. It would be ideal if all patients adhered to investigational medical product (IMP) however many patients become nonadherent due to the length of flickering light in a clinical trial.

The present invention attempts to solve these problems as well as others.

### SUMMARY OF THE INVENTION

Provided herein are systems and methods and for a beta converter to treat Alzheimer's disease and provides increased adherence for therapy sessions.

The systems and methods are set forth in part in the description which follows, and in part will be obvious from the description, or can be learned by practice of the methods, apparatuses, and systems. The advantages of the systems and methods will be realized and attained by means of the elements and combinations particularly pointed out in the appended claims. It is to be understood that both the foregoing general description and the following detailed description are exemplary and explanatory only and are not restrictive of the systems and methods, as claimed.

### BRIEF DESCRIPTION OF THE DRAWINGS

In the accompanying figures, like elements are identified by like reference numerals among the several preferred embodiments of the present invention.

FIG. 1A is a flow chart of the Beta Converter system, according to one embodiment; and FIG. 1B is a flow chart of the Beta Converter system, according to another embodiment.

FIG. 2A is a webpage or display for the Beta Converter system, according to one embodiment; and FIG. 2B is a webpage or display for the Beta Converter system, according to another embodiment.

FIG. 3A is a webpage or display showing the upload bottom to upload a personalized video or photo to the Beta Converter system, according to one embodiment; and FIG. 3B is a webpage or display showing the choose your video, choose your photos, or explore our library options for the Beta Converter system, according to another embodiment.

FIG. 4A is a webpage or display showing the conversion process of the personal video or photo being converted to the 40 Hz video display, according to one embodiment; and FIG. 4B is a webpage or display showing the conversion process of the personal video or photo being converted to the 40 Hz video display, according to another embodiment.

FIG. 5A is a webpage or display showing the download option to view the 40 Hz video display, according to one embodiment; and FIG. 5B is a webpage or display showing the begin session option to view the 40 Hz video display, according to another embodiment.

FIG. 6A is a still image of the video playback of the personal video without the flickering light overlay; FIG. 6B is a still image of the video playback of the personal video transitioning to the flickering light overlay; FIG. 6C is a still image of the video playback of the personal video displaying the flickering light overlay; FIG. 6D is a still image of the video playback of the personal video transitioning to the flickering light overlay; and FIG. 6E is a still image of the video playback of the personal video without the flickering light overlay.

FIG. 7A is a photo library from a user to select for the video playback; and FIG. 7B is a display showing the library of scenes of classic film and photography for the video playback.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the software, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Currently, there are no other products that combine the use of personal memorabilia and footage with the ongoing testing of light therapy in AD. Currently, it is difficult to get patients to adhere to light therapy protocols. The continued pulsing of light does not have any motivational components and may be initially distracting and confusing to patients. Lack of interest could cause patients to not comply with therapy.

In medicine, patient adherence (also compliance, capacitance) describes the degree to which a patient correctly follows medical advice, treatment, or protocol. Most commonly, it refers to medication or drug compliance, but adherence also apply to other situations such as medical device use, self-care, self-directed exercises, or therapy sessions. Both patient and health-care provider affect compliance, and a patient experience is a critical factor in improving compliance. The patient experience is more enjoyable plays a role in patient adherence, whereby greater annoyance contributes to less patient use of the therapy.

The Beta Converter system provides compliance to research protocol, where patients wear headgear and are exposed to disruptive and uncomfortable light therapy will be less inclined to adhere. The Beta Converter system increases cognitive function of patients in the research protocol (including mental arithmetic and short-term memory), reduces of beta amyloid plaque, and overall slowing of progression of mental diseases.

The Beta Converter system is a video display therapeutic comprising methods to convert a video and/or photos to be displayed in a flickering light overlay to a patient or subject. In one embodiment, the flickering light overlay is displayed at a rate of about 40 Hz or about 40 frames per second (fps). In one embodiment, the flickering light is substantially white on a computer display or screen to provide a light therapy. The combination of light therapy with personal memory triggers help stimulate memory while reducing beta amyloid plaques. Additionally, the Beta Converter system ensures compliance of any light therapy protocol in place, since the user has a vested interest in viewing the personalized video or photo.

The Beta Converter interface may be on any video displaying device, including, but not limited to, computer displays, cell phone displays, tablet computers, Virtual Reality headsets, portable video or electronic displays, televisions, monochrome displays, colored displays, electronic projectors and the like. The video display is enabled or operable to electronic hardware to display a video at least at a 40 Hz frame rate. An electronic visual display, informally a screen, is a display device for presentation of images, text, or video transmitted electronically, without producing a permanent record. Electronic visual displays include television sets, computer monitors, and digital signage. An overhead projector displays the presentation of an images, plain text, or video transmitted electronically without producing a permanent record. If a cell phone is used, a wearable mobile headset may be used, with the smartphone inserted into the headset, and head straps securing the cell phone on the user eye line.

The flickering light drives neural activity in the subject's brain. In particular, the flickering light can induce gamma activity in the subject's brain. Gamma activity is neural oscillation with a frequency between about 20 Hz and about 100 Hz, as such, the flickering light may include a frequency between about 20 Hz and about 100 Hz. In some implementations, the gamma activity is neural oscillation in a range between about 20 Hz and about 80 Hz, as such, the flickering light may include a frequency between about 20 Hz and about 80 Hz. Optionally, the gamma activity is neural oscillation at about 40 Hz, as such, the flickering light may include a frequency about 40 Hz. Gamma activity is known in the art and is therefore not described in further detail herein. The gamma activity can be induced in the sensory cortices of the subject's brain. Alternatively or additionally, the gamma activity can be induced in a deep brain region such as the hippocampus, medial temporal lobes, frontal lobes, subcortical structures, thalamus, hypothalamus, or brainstem. The gamma activity can be induced in other brain regions or parts of the nervous system. In one embodiment, inducing gamma activity in the sensory cortices, hippocampus, medial temporal lobes, and/or frontal lobes are provided only as examples.

The video display may be on a webpage or display through a user interface (UI). The user interface (UI) is the point of human-computer interaction and communication in a device. This can include display screens, keyboards, a mouse, voice control, and the appearance of a desktop. It is also the way through which a user interacts with an application or a website. The various types of user interfaces include: graphical user interface (GUI), command line interface (CLI), menu-driven user interface, touch user interface, voice user interface (VUI), form-based user interface, natural language user interface. Elements of a GUI include such things as windows, pull-down menus, buttons, scroll bars and icons. With the increasing use of multimedia as part of the GUI, sound, voice, motion video and virtual reality are increasingly becoming the GUI for many applications. Mobile UIs in mobile applications provides usable, interactive interfaces on the smaller screens of smartphones and tablets and improving special features, like touch controls. A web page (or webpage) is a hypertext document provided by a website or mobile application and displayed to a user in a web browser or on a mobile device.

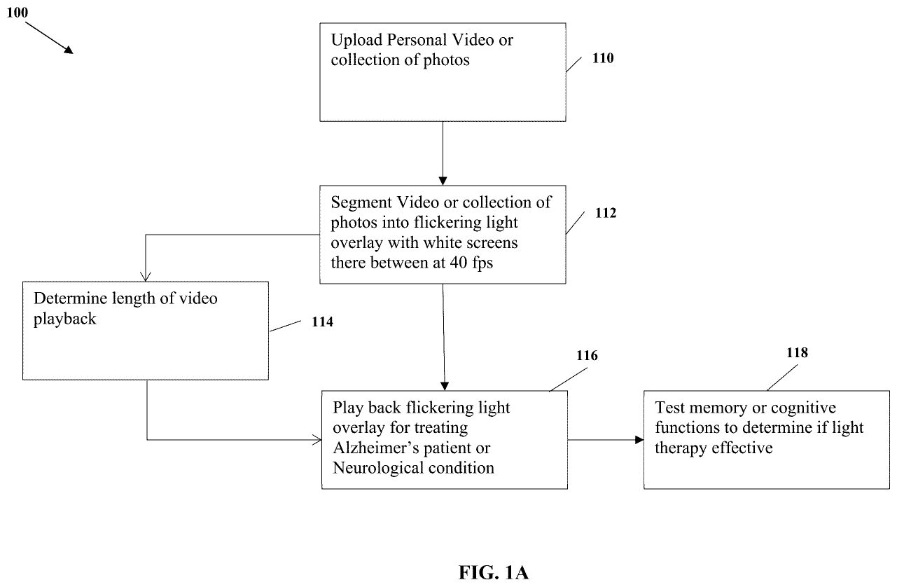

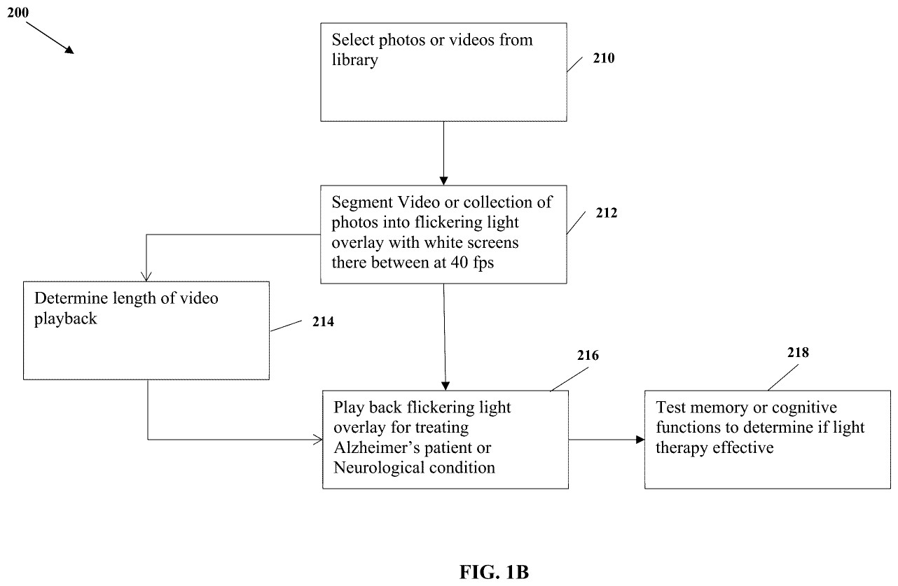

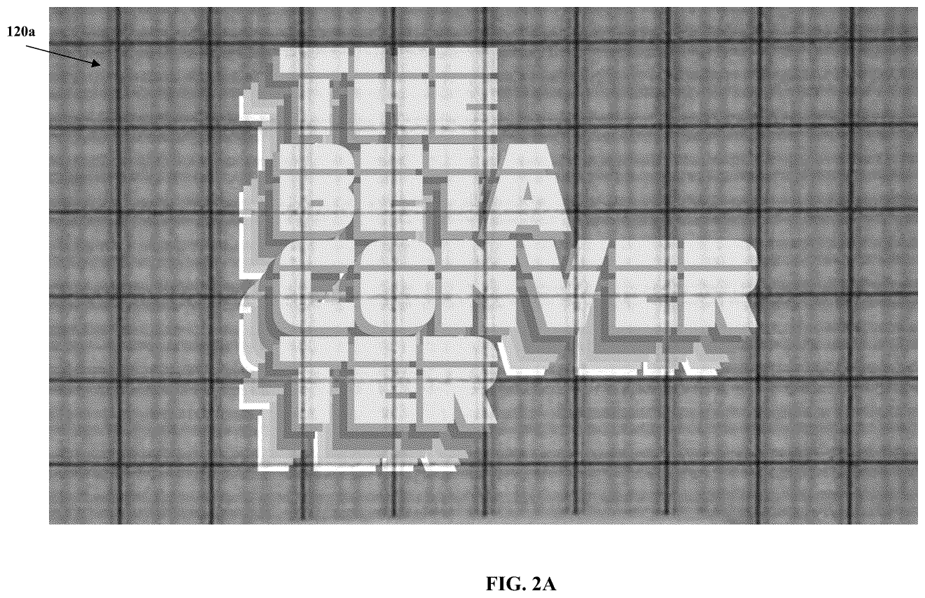

As shown in FIG. 1A, the user uploads the personal video or photos in step 110 to the Beta Converter system 100. In step 112, the Beta Converter system segments the video or collection of photos into flickering light overlay with white screens there between at 40 fps. In step 114, optionally, the Beta Converter determine length of video playback which may be necessary for therapeutic effect. In step 116, the Beta Converter system plays the converted video on the video display including flickering light overlay at 40 fps for treating Alzheimer's patient or neurological condition. In step 118, in an optional step, the Beta Converter system may test memory or cognitive functions to determine if light therapy effective. If the memory or cognitive functions are not effective, the video playback may be repeated.

Uploading refers to transmitting data from one computer system to another through means of a network. Common methods of uploading include: uploading via web browsers, FTP clients, and terminals (SCP/SFTP), or other wireless means of communication. Uploading can be used in the context of clients that send files to a central server. While uploading can also be defined in the context of sending files between distributed clients, such as with a peer-to-peer (P2P) file-sharing protocols, the term file sharing is more often used in this case. Alternatively, the patient or subject may download their videos or photos from a central server that stores the videos or photos, such as Google Photos, Dropbox, Amazon photos, and the like. Downloading is where data is received over a network.

As shown in FIG. 1B, the user selects a video or photos from a library in step 210 to the Beta Converter system 200, according to one embodiment. In step 212, the Beta Converter system segments the video or collection of photos from the library into flickering light overlay with white screens there between at 40 fps. In step 214, optionally, the Beta Converter determine length of video playback which may be necessary for therapeutic effect. In step 216, the Beta Converter system plays the converted video on a video display including flickering light overlay at 40 fps for treating Alzheimer's patient or neurological condition. In step 218, in an optional step, the Beta Converter system may test memory or cognitive functions to determine if light therapy effective. If the memory or cognitive functions are not effective, the video playback may be repeated.

The Beta Converter system is used in combination with clinical trial programs measuring various cognitive abilities suggests. Current studies show after six 30-minute sessions, subjects showed improvements in various cognitive abilities—including mental arithmetic and short-term memory.

The Beta Converter system may be used to treat a neurological disease, injury, condition, or infection (such as, for example, Schizophrenia, Epilepsy, Frontotemporal dementia, vascular dementia, Bipolar disorder, Parkinson's disease, Alzheimer's disease, Autism, Amyotrophic Lateral Sclerosis, Stroke, Traumatic brain injury, bipolar disorder, ischemia reperfusion injury, Multiple sclerosis, and/or Depression) including inflammatory injury due to the neurological disease, injury, condition, or infection in a subject, comprising exposing the subject to a stimulus; the stimulus induces neural activity in the subject's brain and modulates expression of at least one soluble mediator of cellular activity within the subject, and the flickering light stimulus is delivered to the subject for less than one hour.

The total duration of treatment may be from about 1 day to about 4 weeks, or about 1 week to about 10 weeks, or about 1 weeks to about 8 weeks, or about 2 weeks. During this period of time, the subject is started on a program of treatment, and subsequently tested for cognitive related deficits. In some embodiments the treatment with Beta Converter system combined with other substances, as described herein, In some embodiments, the total duration of treatment is about 1 day to about 30 days, or about 5 days to about 10 days, or about 2 days to about 15 days, or about 1 day to about 4 days, or about 12, 13, 14, 15, 16, 17, 18, 19, 20, 21, 22, 23, 24, 25, 26, 27, 28, 29, 30, 31, 32, 33, 34, 35, 36, 37, 33, 39, or 80 days. In some embodiments, the total duration of treatment is about 28 days. In some embodiments, the total duration of treatment is about 56 days. It will be appreciated that these durations are averages, and that individuals using the treatment may vary from the average based on the severity of their symptoms, missing days of treatment, and the like. In some embodiments, the duration of the treatment is based on the individual's symptoms.

Additionally, the light therapy treatment can be delivered to the subject for less than one hour for a therapy session. Optionally, the light therapy treatment can be delivered to the subject for less than about 30 minutes for a therapy session (for example, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 minutes). Optionally, the light therapy treatment can be delivered to the subject for less than about 10 minutes for a therapy session (for example, 10, 9, 8, 7, 6, 5, 4, 3, 2, 1 minutes). Optionally, the light therapy treatment can be delivered to the subject for less than about 5 minutes for a therapy session (for example, 5, 4, 3, 2, 1 minutes).

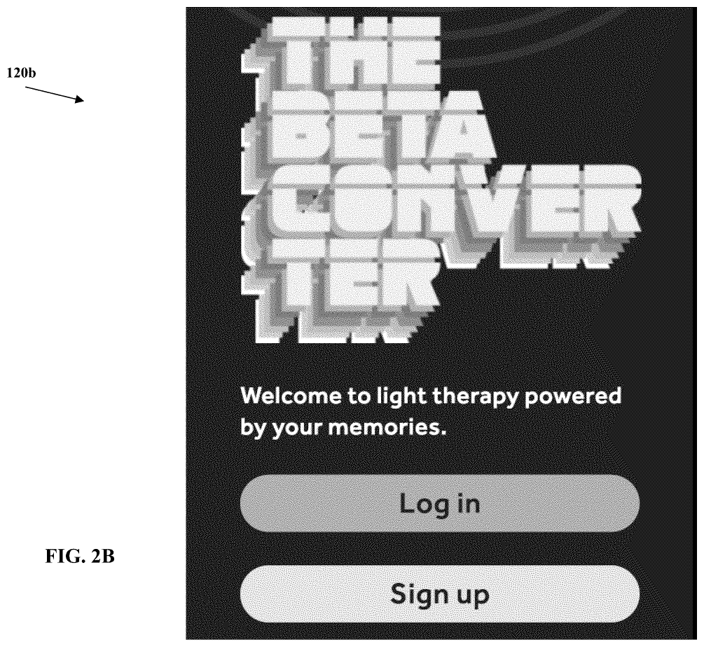

As show in FIG. 2A, the user is provided a main webpage 120a providing the Beta Converter system 100 and links to additional steps for processing the flickering light stimulus, according to one embodiment. As show in FIG. 2B, the user is provided a main webpage 120b providing the Beta Converter system 100 and links to log in or sign up for the light therapy and processing the flickering light stimulus, according to one embodiment. A user may log in or sign up by providing their email address, mobile number, and then entering a user specific password, confirming the user specific password and agreeing to the terms of use. A user may sign up by providing their first name, last name, date of birth, and user specific password.

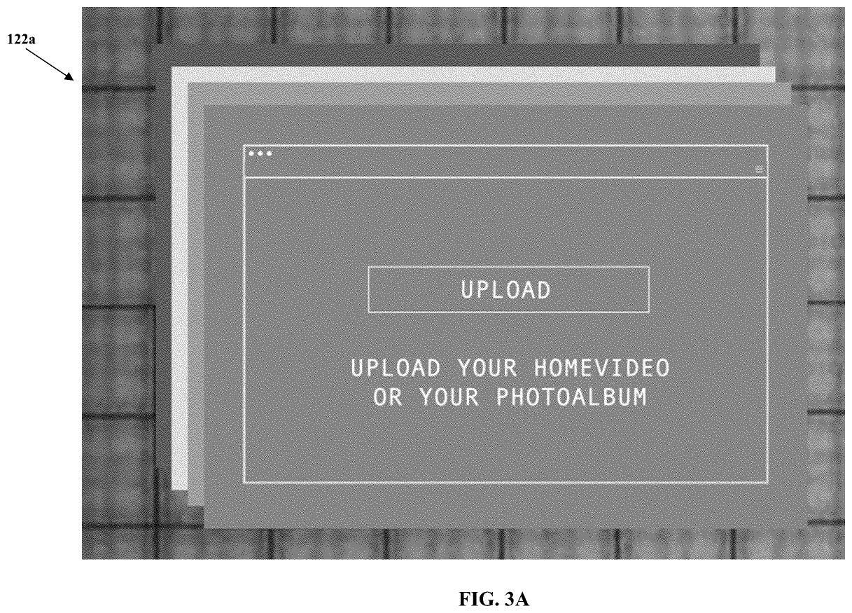

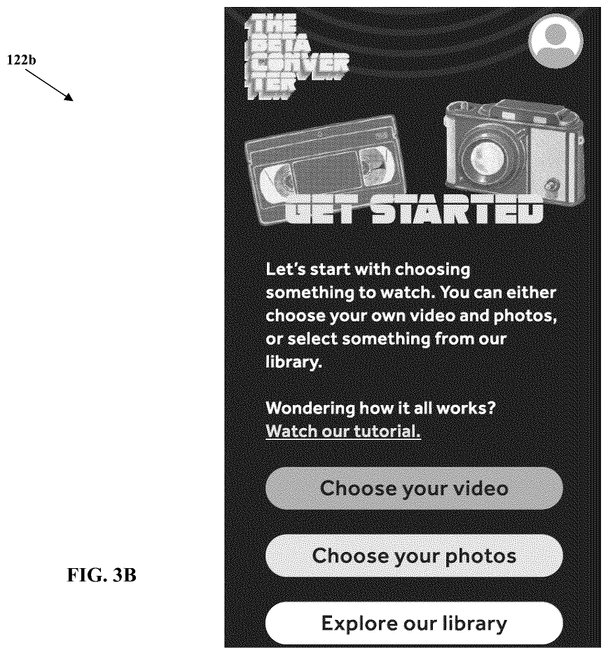

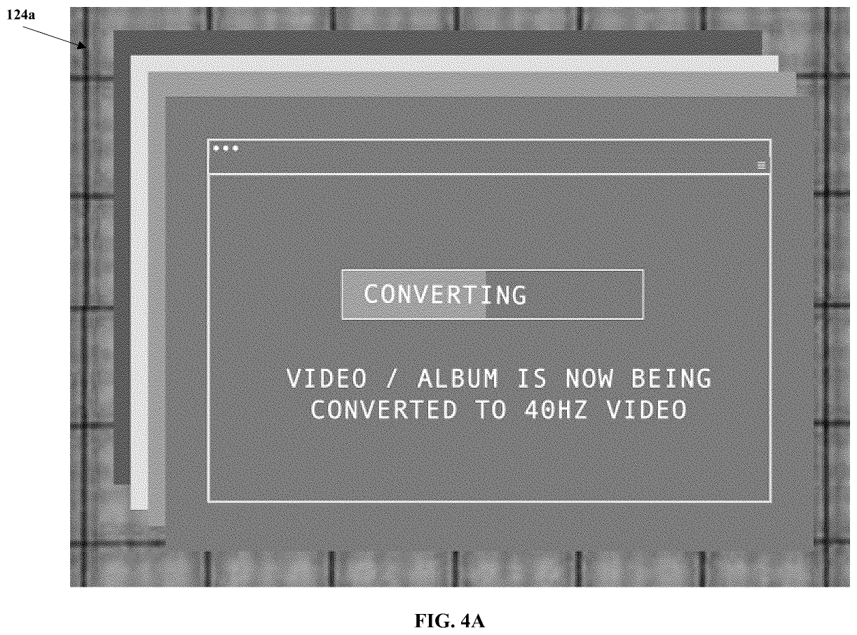

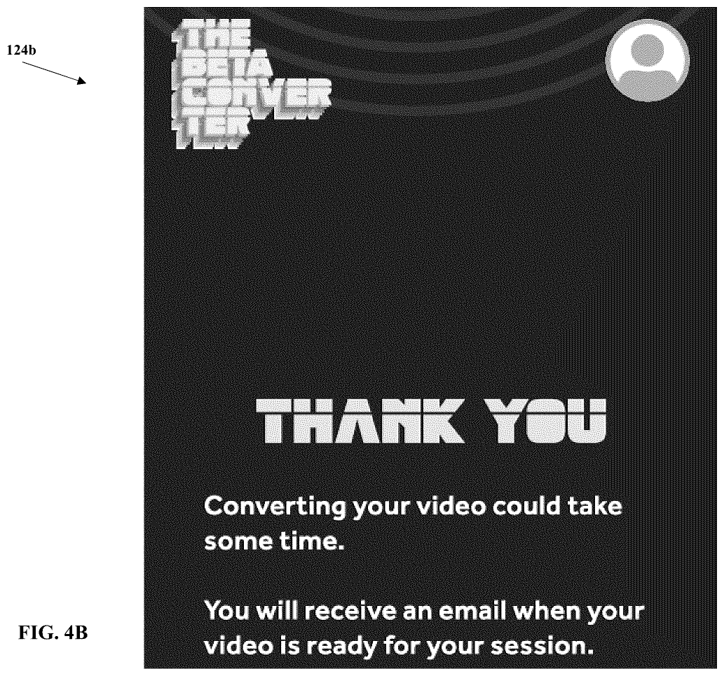

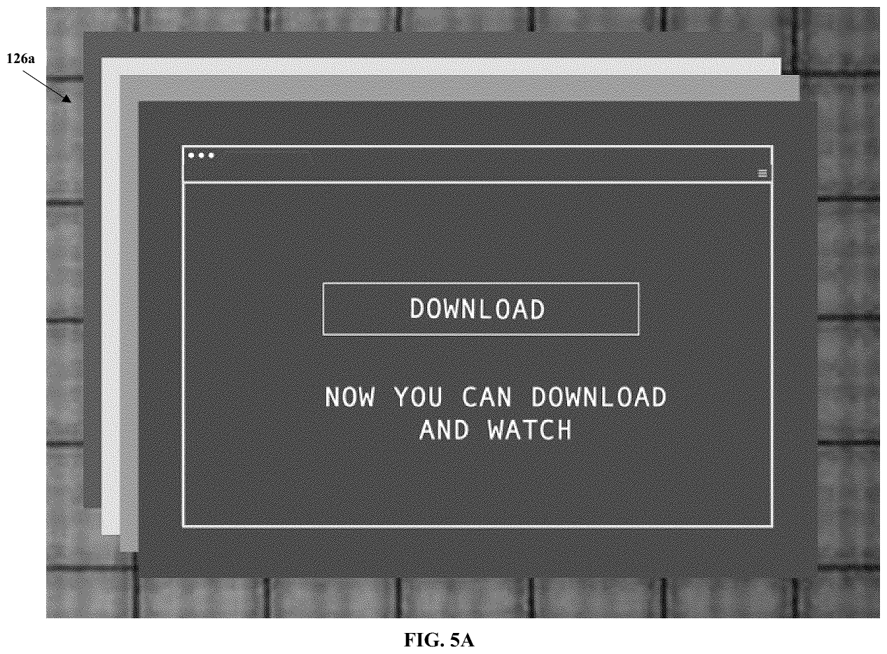

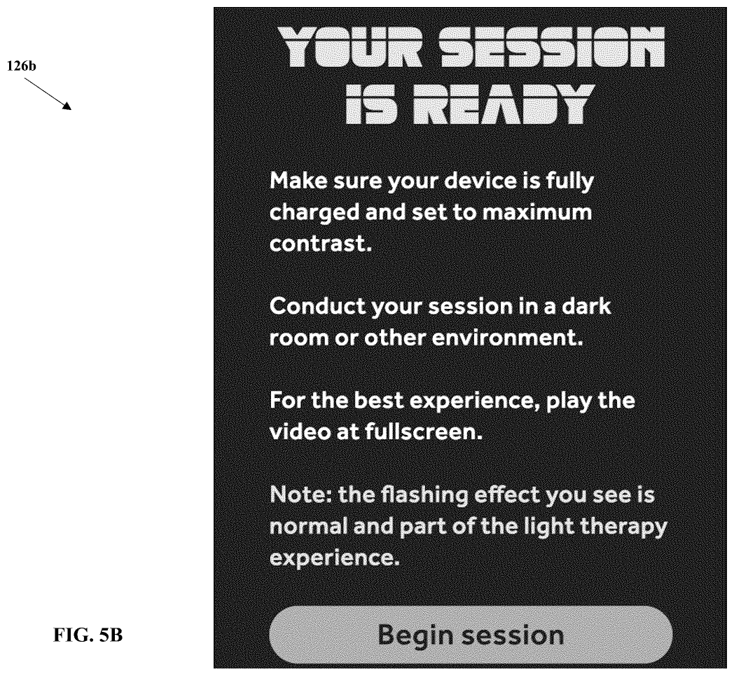

As shown in FIG. 3A, the Beta Converter provides a webpage 122a for the user to upload the personal video or photos. As shown in FIG. 3B, the Beta Converter provides a webpage 122b for the user to choose a video, choose a photo, or explore the library of videos and photos. There is also an option to watch a tutorial to see how the Beta Converter system works. In one embodiment, the user may name the session with a specific video or photo selected. As shown in FIG. 4A, the Beta Converter provides a webpage 124a to convert the personal video or photos to the 40 Hz video to display the light therapy stimulus, according to one embodiment. As shown in FIG. 4B, the Beta Converter provides a webpage 124b showing the converting the personal video or photos to the 40 Hz video to display the light therapy stimulus, according to one embodiment. In one option, the user may receive an email or text notification when the video or photo conversion session is ready. As shown in FIG. 5A, the Beta Converter provides a webpage 126a to download or display the video including the light therapy stimulus to be played back to the user on the computer display, according to one embodiment. As shown in FIG. 5B, the Beta Converter provides a webpage 126b to begin the video session including the light therapy stimulus to be played back to the user on the user's cell phone or portable electronic device, according to one embodiment. The Beta Converter may provide instructions for the user, ensuring the user's electronic device is fully charged, the video playback is conducted in a dark room, and to play the video on the full screen mode. The Beta Converter may provide a note that the flashing effect is a normal part of the light therapy experience.

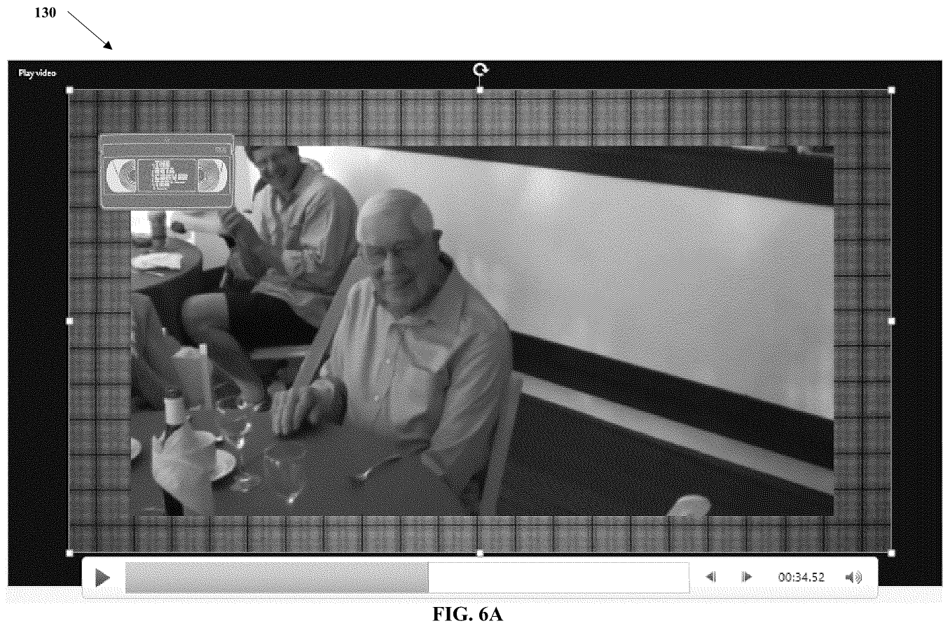

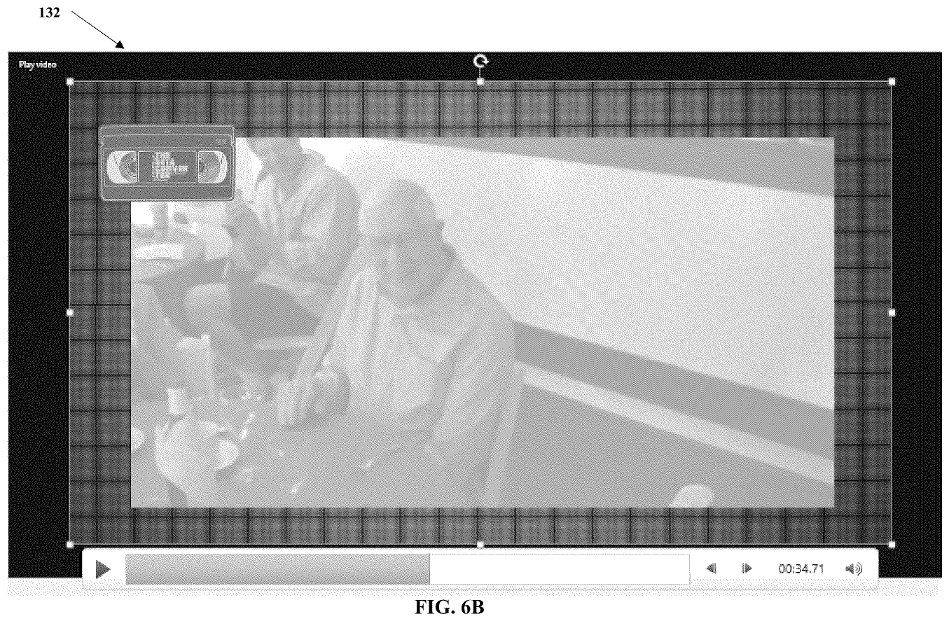

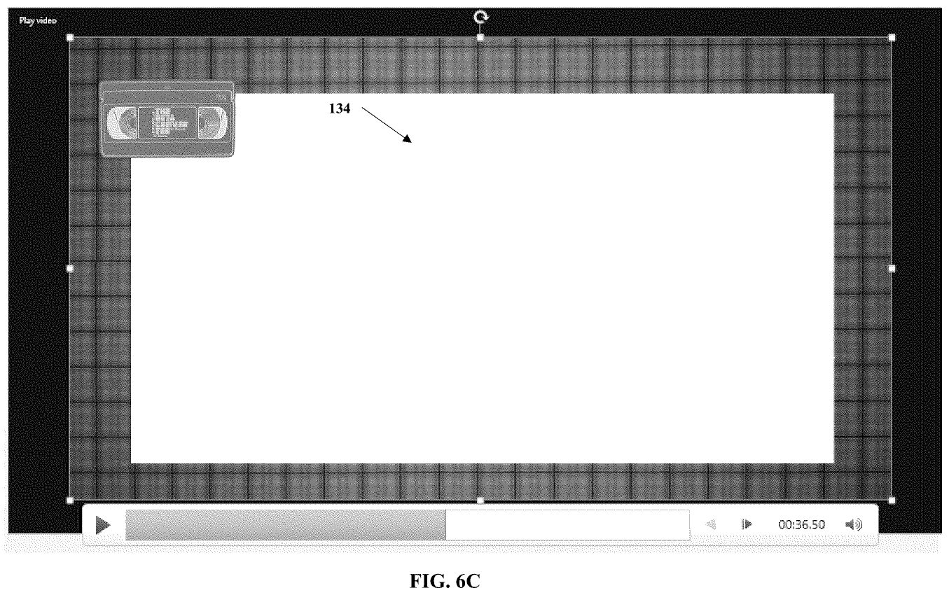

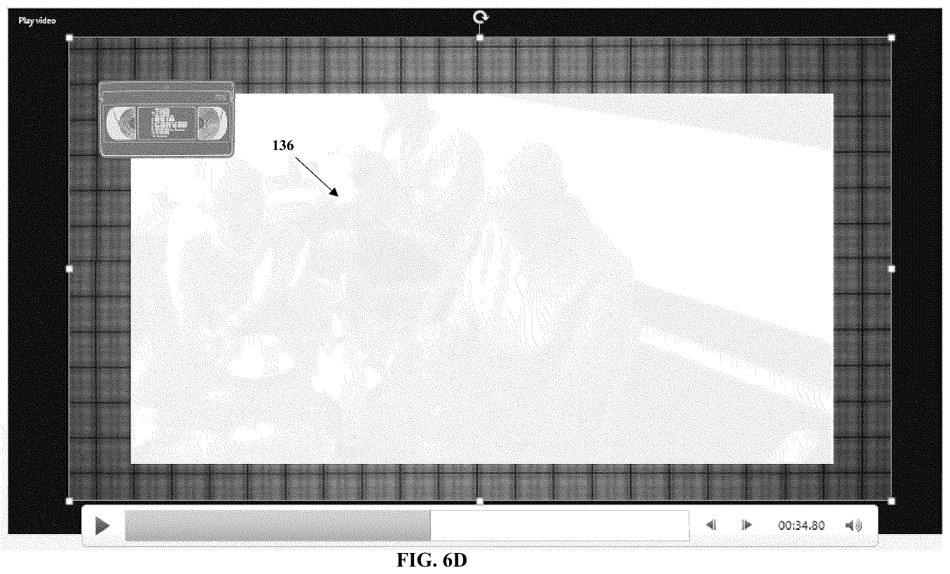

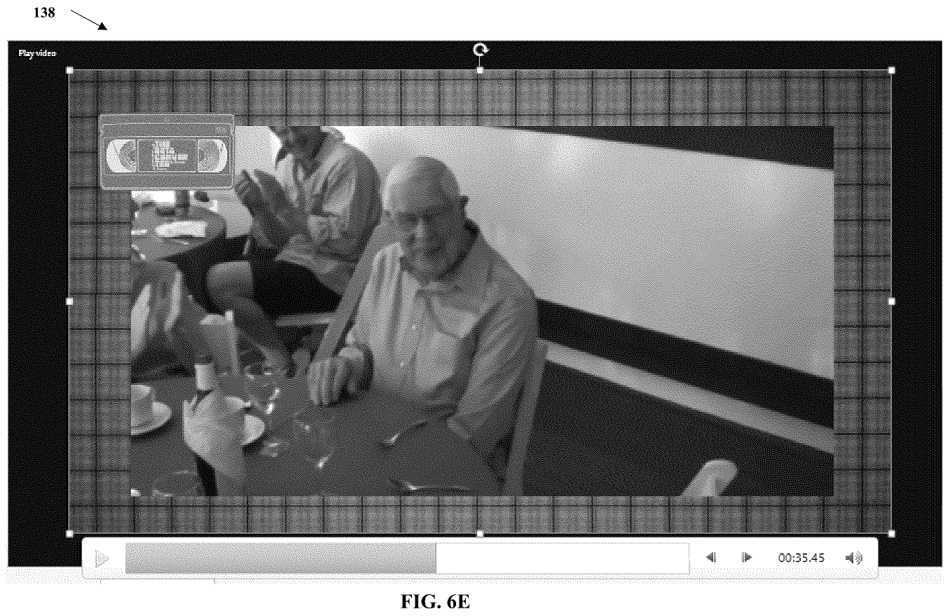

As shown in FIG. 6A, the user plays the video and the initial image is displayed on webpage 130 including the original image and the original audio from the video. As shown in FIG. 6B, the subsequent image and audio is a fading white overlay after 5.75 milliseconds which then transitions to the complete white image display 134, as shown in FIG. 6C. As shown in FIG. 6D, the subsequent image and audio is a fading white overlay within 5.75 milliseconds from the complete white image display. As shown in FIG. 6E, the subsequent image is a complete photo from the video file and audio. The complete cycle takes about 12.5 milliseconds, according to one embodiment.

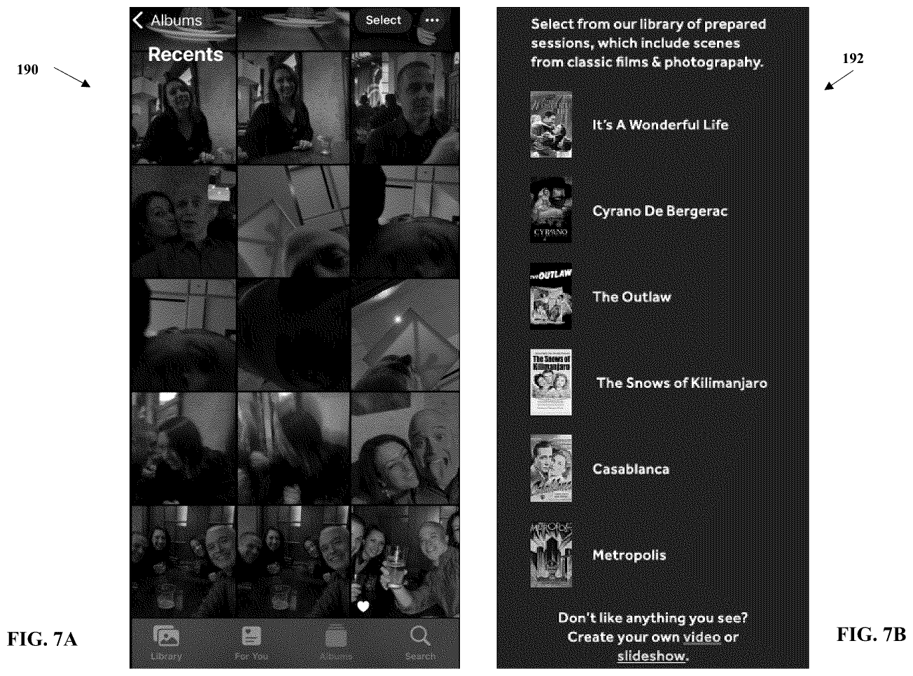

As shown in FIG. 7A, the user may select a photo from their photo library 190 or the user may select a video from a video library 192, as shown in FIG. 7B.

Beta Converter Stimulus

The Beta Converter system and methods comprise treating a neurological disease, injury, condition, or infection of any preceding aspect, wherein the flickering light overlay comprises a 40 Hz display of white screen with personal videos or photos and wherein the neurological condition comprises inflammatory damage resulting from aging, traumatic brain injury, stress, schizophrenia, and/or depression. In one aspect, disclosed herein are methods of treating a neurological disease, injury, condition, or infection of any preceding aspect, wherein the stimulus comprises a display of a white screen at a rate between about 20 Hz to about 80 Hz.

An example for driving electrical activity of neurons to alter signaling that governs inflammation, cell survival, plasticity and other cell functions is described herein. The flickering light overlay is used to induce gamma neural activity in the visual cortex of a brain, and affects inflammatory signals. The effects of flicker on numerous intracellular inflammatory signals is shown in the visual cortex. The flickering light overlay at 40 Hz triggers a rapid spike (<5 min) in a subset of inflammatory pathways followed by increased production of inflammatory cytokines known to regulate microglia within an hour. The flickering light overlay drives neural activity (and in some implementations driving neural activity non-invasively) and drives molecular signaling in the brain that controls inflammation. Moreover, these inflammatory pathways and the downstream genes control diverse beneficial cellular functions, including survival, proliferation, differentiation, plasticity, and neurogenesis, among others. Therefore, the flickering light overlay modulate brain inflammation to treat many brain diseases, brain injury, infection, and the effects of normal brain aging. For one embodiment, the flickering light overlay drives neural activity to modulate inflammatory signaling can be used to treat neurodegenerative diseases (e.g., Alzheimer's disease). In another embodiment, the flickering light overlay drives neural activity to modulate inflammatory signaling can be used to treat disorders that involve inflammatory signaling (e.g., schizophrenia), the brain's immune responses, and/or neural activity.

The flickering light overlay at gamma frequencies are used to drives strong rhythmic neural activity in visual cortex. The visual cortex is known to be highly sensitive to light flicker. The flickering light overlay triggers a rapid spike (e.g., in <5 min) in a subset of pathways (e.g., MAPK, NF-κB (nuclear factor kappa-light-chain-enhancer of activated B cells)) followed by increased production of cytokines known to regulate microglia within an hour. Over a time course of flickering light overlay exposure, neural electrical activity dramatically increases, suggesting neural circuits are undergoing plasticity. These gamma oscillations trigger common intracellular pathways that induce genes which govern both immune function and synaptic plasticity.

The flickering light overlay can be produced with light (e.g., white light), and/or an auditory stimulus can be produced with sound. Optionally, the stimulus can be sensory flicker used to induce gamma activity. For example, the stimulus can be a 20 Hz sensory flicker stimulus or white-light at 4,000K flicker has been shown to stimulate the neuroprotective effect. Alternatively, the stimulus can be a 40 Hz sensory flicker stimulus. Visual flicker stimulus can be produced by flashing a light at a desired frequency (e.g., 12.5 millisecond (ms) ON every 25 ms for 40 Hz, 25 ms ON every 50 ms for 20 Hz). This disclosure contemplates that duty cycles between 4-50% can be used for the visual flicker stimulus. It should be understood that the color and/or parameters (e.g., frequency, period, duty cycle, etc.) of the visual flicker stimulus are provided only as examples and can have other characteristics/values while still inducing gamma activity.

In other implementations, the flickering light overlay is coupled with an auditory flicker. Auditory sensory flicker can drive gamma oscillations in the Hippocampus. For one embodiment, an auditory flicker stimulus can optionally be produced by sounding a 1 ms long 10 kHz tone every ms. The tonal frequency and/or parameters (e.g., frequency, period, duty cycle, etc.) of the auditory flicker stimulus can have other characteristics/values while still inducing gamma activity. In other embodiments, the sensory flicker can be combined visual and auditory flicker. For example, a combined visual and auditory flicker stimulus can optionally be produced by flashing a light and sounding a tone every 25 ms.

In one embodiment, the Beta Converter is a web-based application with a customized video filter designed to research specifications. Content is uploaded, processed, and exported for viewing. In another embodiment, the Beta Converter is a software based application incorporated into a computer, smart phone, tablet, and the like.

The various video-to-flickering light overlay techniques, methods, and systems described herein can be implemented in part or in whole using computer-based systems and methods. Additionally, computer-based systems and methods can be used to augment or enhance the functionality described herein, increase the speed at which the functions can be performed, and provide additional features and aspects as a part of or in addition to those described elsewhere in this document. Various computer-based systems, methods and implementations in accordance with the described technology are presented below.

The video stream may be in format of (but not limited to): Advanced Video Codec High Definition (AVCHD), Audio Video Interlaced (AVI), Flash Video Format (FLU), Motion Picture Experts Group (MPEG), Windows Media Video (WMV), or Apple QuickTime (MOV), h.264 (MP4).

The Beta Converter system and method of generates content from video files or photo files such as to display a flickering light overlay found in the images of the video files. In an embodiment, the video-to-flickering light overlay engine can perform the functions directed by programs stored in a computer-readable medium. That is, the embodiments may take the form of a hardware embodiment (including circuits), a software embodiment, or an embodiment combining software and hardware. The present invention can take the form of a computer-program product that includes computer-useable instructions embodied on one or more computer-readable media.

System

For exemplary purposes only, and not to limit one or more embodiments herein, a video file is segmented into video data and audio data segments. A data pipeline with a Video Input/Output, extract sequences of image frames and can warehouse compressed images in a distributed data store as image frame data. A distributed computation engine can be dedicated to image pre-processing for performing image segmentation. Processed data can be sent to one or more machines that can combine and/or sort segmented video and audio data in a time-ordered fashion. Similarly, the Audio Input/Output represents a data pipeline for downloading or warehousing in a distributed file system. The audio can be, for example but not limited to WAV, .MP3, or other known formats.

In other embodiment, the Beta Converter system can be included, for example, a laptop, a tablet, a mobile phone, a personal digital assistant (PDA), a keyboard, a display monitor with or without a touch screen input, and an audiovisual input device. In another implementation, the peripheral devices may themselves include the functionality of the general-purpose computer. For example, the mobile phone or the PDA may include computing and networking capabilities and function as a general purpose computer by accessing a network and communicating with other computer systems.

The server can include the general purpose computer discussed above. The network includes, for example, the Internet, the World Wide Web, WANs, LANs, analog or digital wired and wireless telephone networks (e.g., Public Switched Telephone Network (PSTN), Integrated Services Digital Network (ISDN), and Digital Subscriber Line (xDSL)), radio, television, cable, or satellite systems, and other delivery mechanisms for carrying data. A communications link can include communication pathways that enable communications through one or more networks.

In some embodiments, a video-to-content engine can be embodied in a server or servers. The Beta Converter system requests an application relating to the video stream. The servers perform the video data and audio data segmented process in parallel. This method allows real-time video data and audio data segmented conversion.

The Beta Converter system communicates with the server via the network for video stream application. The Beta Converter system engine can generate the video. The server then generates data based on the continuous playback. In some embodiments, the server adds a specific symbol, image, frame, or a specific time in the video stream to determine if the user is viewing the video. The specific symbol, image, frame, or the specific time in the video stream can be selected based on the video text.

Syncing to Other Systems for Personalized Content

In one embodiment, the Beta Converter system can sync or provide an option for the user to provide access to third party websites, servers, or storage system to access personalized video files or photo files. Such third party websites, servers, or storage system include but are not limited to: iCloud, Google Photos, Amazon photos, Dropbox, Box, and the like. The user provides security access to these third party websites, servers, or storage systems to upload and create the segmented video file.

### EXAMPLES

### Example 1: Beta Converter Workshop

Objective

The workshop will provide user feedback (patients and caregivers) about usability on Beta Converter as described herein, which is a digital product to help improve cognition and memory in people with cognitive impairment.

Procedure

The workshop will work with members and caregivers to identify gaps and opportunities in the design of the Beta Converter.

Participants will have a pre-assignment to do prior to workshop day, conducted with their Care Partner: 1) Pre-load Beta Converter on a mobile device that has the ability to process uploading and converting of home video and images; 2) Pre-upload digital files to convert using the Beta Converter; and 3) Bring to workshop digital album of personal photos AND/OR videos

The data collected in the form of qualitative feedback, in the form of the focus group includes product usability, product design, onboarding, ancillary equipment design, adherence, and the role of the caregiver.

Product Usability: Overall, how was using the product compared to the Halo?

Product Design: How was the functionality of the digital product itself?

Onboarding: How might we better onboard users to the product?

Ancillary Equipment Design: Does this work for you, now and in the future?

Adherence: Would you be more likely to use this vs the Halo?

Role of the Caregiver: Can you do this on your own? Would you prefer that, or want the help from your caregiver?

Individuals will sign consent for allowing recording of focus groups for research and internal purposes only. Participants will also have the option of signing a standard waiver for broader use of the footage.

Research does not require subjects to be deceived. There will be full transparency into the digital device and the workshop intentions.

As a courtesy, the general results of the study will be provided with no individual results.

The light from the Beta Converter may induce stress and some of the subjects may have been exposed to the Beta Converter light therapy prior to the Workshop. Subjects will be closely monitored as they use the Beta Converter, if they have a negative reaction, the therapy session will be immediately discontinue.

Members with Mild cognitive impairment (MCI), or active user of the Beta Converter may be selected as candidates. Participants should be able to come with a Care Partner, with a personal Mobile Device capable of displaying audio and video, access to the internet, with sufficient processing ability to stream at a reasonable speed.

Participants may undergo venous blood draws, lumbar punctures, magnetic resonance imaging (MRI) scans, electroencephalograms (EEGs), and cognitive testing at baseline (see Supplementary Methods in supporting information for details). All procedures except for cognitive testing may be repeated at midpoint and at the end of study. Lumbar punctures performed within the prior 6 months may be used for baseline when available. Participants with MCI due to AD, as evidenced by positive cerebrospinal fluid (CSF) biomarkers of AD.

Clinical Trial Adherence

Adherence rates may average about 95% on average with all participants having adherence rates greater than 90% during the main phase of the study. Adherence rates per week may be above 90% on average over 8 weeks

Default Mode Network Functional Connectivity after 8 Weeks of Daily Beta Converter Use

During resting state, functional connectivity in the default mode network is lower in AD patients compared to healthy controls. Therefore, to assess the Beta Converters effects on resting state and regional connectivity relevant to AD, functional connectivity using fMRI between posterior cingulate cortex (PCC) and precuneus (PCu) and betweenPCCand medial prefrontal cortex (mPFC) nodes of the default mode network may be assessed. Functional connectivity between PCC and PCu may significantly increase after 8 weeks of daily Beta Converter use, but not in other groups (P-values>0.25). No significant differences may be found in the functional connectivity between PCC and mPFC across any exposure period. The functional connectivity between PCC and PCu, which is weakened in AD, may be strengthened after 8 weeks of daily Beta Converter use.

Changes in CSF Aβ and Tau Levels after 4 or 8 Weeks of Daily Beta Converter Use

To assess potential changes in AD biomarkers, levels of Aβ42, p-tau, t-tau, and the ratio t-tau/Aβ42 may be assessed in the CSF of participants before and after 4 weeks of no Beta-converter use, 4 weeks of daily Beta converter use, and 8 weeks of daily Beta converter use. No significant differences in the changes in Aβ42, p-tau, t-tau, or t-tau/Aβ42 ratio after 4 or 8 weeks of daily Beta converter use may be seen.

Altered Levels of Cytokines and Immune Factors in CSF after 8 Weeks of Daily Beta Converter Use

If Beta converter use alters levels of cytokines and other immune factors as evidence of immune engagement in humans. To isolate the effects of Beta Converter use on immune factors regardless of differences in individuals' underlying Aβ42 and p-tau pathology, effects related to Aβ42 and p-tau using a linear model may be adjusted. A partial least squares discriminant analysis (PLSDA) may be used to identify a latent variable 1 (LV1), that distinguished among pre-Beta Converter use, 4-week, or 8-week Beta converter use groups. LV1 may be made up of a weighted profile of cytokines and immune factors, with negative values indicating a decrease in that cytokine after 8 weeks of Beta converter use. Using paired t-tests comparing samples from the same participant at different time points, LV1 may increase after 8 weeks compared to pre-Beta converter use. The cytokine TWEAK (tumor necrosis factor-related weak inducer of apoptosis) may significantly decrease after 8 weeks of Beta converter use. Several factors may show trends of downregulation after 8 weeks of Beta Converter use including transforming growth factor alpha (TGF-α), macrophage inflammatory protein 1β (MIP-1β), Delta and Notch-like epidermal growth factor receptor (DNER), and interleukin (IL)-5. Changes in the immune profile in CSF may show trends toward downregulation of immune factors suggesting engagement of the neuroimmune system after chronic exposure to Beta converter use.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of ensuring compliance of a light therapy protocol comprising: uploading a personal video or a photo in a Beta Converter system; segmenting the personal video or the photo into a flickering light overlay with white screens there between at 40 fps; determining length of video playback for the light therapy protocol; playing the converted video including flickering light overlay at 40 fps for treating a mental disease.

2. The method of claim 1, further comprising increasing cognitive function of patients in the research protocol; and reducing of beta amyloid plaque and slowing of progression of mental diseases.

3. The method of claim 2, wherein the length of video playback is at least 30-minutes.

4. The method of claim 3, wherein the mental disease, injury, condition, or infection is selected from the group consisting of: Schizophrenia, Epilepsy, Frontotemporal dementia, vascular dementia, Bipolar disorder, Parkinson's disease, Alzheimer's disease, Autism, Amyotrophic Lateral Sclerosis, Stroke, Traumatic brain injury, bipolar disorder, ischemia reperfusion injury, Multiple sclerosis, Depression, and inflammatory injury due to the neurological disease, injury, condition, or infection in a subject.

5. The method of claim 4, further comprising testing memory or cognitive functions to determine if light therapy protocol is effective.

6. A Beta Converter system comprising: a video display converting a video and/or photos to be displayed in a flickering light overlay; displaying the flickering light overlay at a rate of about 40 hz or about 40 fps; wherein the flickering light is substantially white on the video display or screen to provide a light therapy; and the combination of light therapy with personal memory triggers help stimulate memory while reducing beta amyloid plaques.

7. The Beta Converter system of claim 6, wherein the Beta Converter system ensures compliance of any light therapy protocol.

8. The Beta Converter system of claim 7, wherein the Beta Converter system treats a neurological disease, injury, condition, or infection of any preceding aspect, and displaying the white screen at a rate between about 20 Hz to about 80 Hz.
